(19) United States
(12) Patent Application Publication
(10) Pub. No.: US 2024/0345080 A1
(43) Pub. Date: Oct. 17, 2024

(54) BIOCHIP SENSOR FOR DETECTING OF MOLECULES

(71) Applicant: BAR-ILAN UNIVERSITY, Ramat Gan (IL)

(72) Inventors: Rachela POPOVTZER, Givat Shmuel (IL); Shir HOCHWALD LIBER, Petah Tikva (IL); Omry KOREN, Yesud Hama'ala (IL); Ehud BANIN, Tel Aviv (IL); Yossi BEN DAVID, Alfei Menashe (IL)

(21) Appl. No.: 18/748,675

(22) Filed: Jun. 20, 2024

Related U.S. Application Data
(63) Continuation-in-part of application No. PCT/IL2022/051364, filed on Dec. 21, 2022.
(60) Provisional application No. 63/292,502, filed on Dec. 22, 2021.

(51) Int. Cl. G01N 33/554; G01N 33/543

(52) U.S. Cl. CPC G01N 33/554; G01N 33/5438; G01N 2333/21; G01N 2333/245; G01N 2333/938; G01N 2800/065

(57) ABSTRACT

Provided herein are biochip, system and method for detection of a marker molecule in a sample, the biochip including one or more chambers, each chamber containing a synthetic biosensor module and a detection module, wherein the synthetic biosensor module comprises a genetically modified bacteria expressing a receptor capable of binding the marker molecule, and a reporter gene, wherein expression of the reporter gene is induced by the binding of the marker molecule to the receptor; and wherein the detection module produces an output signal indicative of presence of the marker molecule in the sample.

### CROSS REFERENCE TO RELATED APPLICATIONS

This application is a Continuation-in-Part of PCT Patent Application No. PCT/IL2022/051364, having International filing date of Dec. 21, 2022, which claims the benefit of priority of U.S. Provisional Patent Application No. 63/292,502, filed Dec. 22, 2021, the contents of which are all incorporated herein by reference in their entirety.

### FIELD OF THE INVENTION

Provided herein are biochips comprising genetically modified bacteria configured to detect various molecules such as but not limited to quorum sensing signaling molecules and methods for using same for detecting and/or monitoring diseases.

### BACKGROUND OF THE INVENTION

The human microbiota is the aggregate of microorganisms that resides on the surface and in deep layers of skin, in the saliva and oral mucosa, in the conjunctiva, and in the gastrointestinal tracts. The human body consisting of about 100 trillion cells and carries about ten times as many microorganisms in the intestines. The microbiome is reckoned to have around 3 million functional genes compared to 23,000 genes in human beings. The far larger genome of the microbiome has correspondingly greater capabilities in modulating human health and well-being.

Research suggests that the relationship between gut flora and humans is not merely commensal (a non-harmful coexistence), but rather a symbiotic relationship. These microorganisms perform a host of useful functions, such as fermenting unused energy substrates, training the immune system, forming a protective mucosal biofilm, preventing growth of harmful, pathogenic bacteria, regulating the development of the gut, producing vitamins for the host (e.g., biotin and vitamin K), producing hormones to direct the host to store fats, producing signaling molecules that promote homeostasis, metabolizing drugs, xenobiotics, and the like.

Invasion by pathogens can constitute various illnesses such as Irritable Bowel Syndrome (IBS), C. difficile infection (CDI), diarrhea, pseudomembranous colitis and others. However, the precise mechanism of this microbial influence in disease pathogenesis remains elusive and is now a major research focus.

To date, detection of bacterial related molecules in the human gut microbiome is based largely on chromatography and mass spectrometry.

Hence, there is an unmet need for a non-invasive, sensitive and reliable system for detecting biomarker molecules, in real-time.

### SUMMARY OF THE INVENTION

Disclosed herein is a synthetic biology-based biochip sensor, comprising genetically modified bacteria configured to detect bacterial biomarker molecules, (e.g. siderophores), as well as molecules secreted by an infected host such as markers for inflammation (e.g. lipocalin), thereby enabling quantitative, sensitive, and real-time detection of the molecules which in turn may facilitate non-invasive early diagnosis and monitoring of diseases, including, but not limited to, gastrointestinal diseases. As detailed below, the design of the biochip incorporates synthetic bacteria together with advanced electronics and wireless communication.

Extensive studies over the past few years show a strong connection between the human gut microbiome e population and the etiology of a variety of diseases, ranging from gastrointestinal diseases, cancer, cardiovascular and respiratory disease, to neurodegenerative and mental disorders.

Advantageously, the herein disclosed biochip includes a) a “synthetic biosensor module” which includes bacteria genetically modified to recognize specific molecules and in response thereto to produce a reporter molecule or reporter signal allowing quantitative identification of the molecules and b) a “quantitative detection module” which includes one or more sensors configured to detect the reporter gene and its levels

According to some embodiments, there is provided biochip for detection of a molecule associated with a gastrointestinal disease or disorder in a sample, the biochip comprising one or more chambers, each chamber comprising a synthetic biosensor module and a detection module, wherein the synthetic biosensor module comprises a genetically modified bacteria expressing a receptor capable of binding the molecule, and a reporter gene, wherein expression of the reporter gene is induced by the binding of the molecule to the receptor; and wherein the detection module comprises a reporter gene substrate, wherein interaction between the expressed reporter gene and the reporter gene substrate produces an output signal indicative of presence of the molecule in the sample.

According to some embodiments, detection module is a quantitative detection module and/or qualitative detection module.

According to some embodiments, the intensity and/or strength of the output signal is indicative of the concentration of the molecule in the sample.

According to some embodiments, the biochip comprises at least two chambers, each chamber comprising a detector in the form of genetically modified bacteria expressing a receptor capable of binding a different molecule associated with the same or a different gastrointestinal disease or disorder. According to some embodiments, the ratio between the output signal of each of the at least two chambers is indicative of a ratio between different bacterial populations in the sample. According to some embodiments, the combined outputs of the at least two chambers is indicative of a specific bacterial population in the sample and/or of the status of gut.

According to some embodiments, the biochip comprises at least two chambers, each chamber configured to sequentially receive samples, e.g. once an hour, once a day, or once a week, thereby allowing monitoring a concentration of bacterial populations over time and/or a severity of a GI disease, without requiring exchanging/replacing the biochip.

According to some embodiments, the detector (i.e. the genetically modified bacteria) may be immobilized so as to enable cleansing of the one or more chambers between samples, thereby allowing reliable readings during multiple uses of the detector.

According to some embodiments, the biochip may be incorporated into a sampling tube. As a non-limiting example, the sampling tube may be configured to periodically receive samples from food production lines, water reservoirs etc. In the food industry there is a great need for real-time and fast technology for the detection of a bacterial contamination of the food and lines. Currently, it takes expensive methods such as PCR tests to verify bacterial food contamination or plate cultures which are time consuming (>24 h). The herein disclosed biosensor offers a low-cost, fast and real-time method for very early detection of bacterial contamination in food and water.

According to some embodiments, the molecule is a quorum sensing molecule. According to some embodiments, the quorum-sensing signaling molecule is an N-acyl-L-homoserine lactone (AHL) found primarily in Gram negative bacteria. According to some embodiments, the quorum-sensing signaling molecule is an autoinducer 2 (AI-2) produced and recognized by many Gram-negative and Gram-positive bacteria. According to some embodiments, the quorum sensing molecule is selected from C4HSL, C6-oxo-HSL, 3-oxo-C12-HSL, C12-HSL, C10-HSL, 3-oxo-C10-HSL AI-2, or combinations thereof.

According to some embodiments, the detector is a C4-HSL receptor, a C6-oxo-HSL receptor, 3-oxo-C12-HSL receptor, C12-HSL receptor, C10-HSL receptor, 3-oxo-C10HSL receptor, a QscR, AI-2 receptor, or any combination thereof.

According to some embodiments, the molecule is a siderophore. According to some embodiments, the molecule is an inflammation marker, such as but not limited to lipocalin and/or calprotectin.

According to some embodiments, the output signal is an electric signal.

According to some embodiments, the reporting agent is selected from the group consisting of: beta-galactosidase, glutathione-S-transferase (GST), c-myc, 6-histidine (6×His), maltose binding protein (MBP), influenza A virus haemagglutinin (HA), and GALA. Each possibility is a separate embodiment. According to some embodiments, the reporter gene is beta-galactosidase, and the reporter gene substrate is p-aminophenyl-β-D-galactopyranoside.

According to some embodiments, the biochip further comprises a DNA probe encoding a transporter of molecule.

According to some embodiments, the molecule is associated with a gastrointestinal disease or disorder.

According to some embodiments, there is provided microbiome analysis system comprising a biochip for detection of a molecule associated with a gastrointestinal disease or disorder in a sample, and a processing module. According to some embodiments, the biochip comprises one or more chambers, each chamber comprising a synthetic biosensor module and a detection module. According to some embodiments, the synthetic biosensor module comprises a genetically modified bacteria expressing a receptor capable of binding the molecule, and a reporter gene, wherein expression of the reporter gene is induced by the binding of the molecule to the receptor. According to some embodiments, the detection module comprises a reporter gene substrate, wherein interaction between the expressed reporter gene and the reporter gene substrate produces an output signal. According to some embodiments, the processing module is configured to receive the output signal and to provide an indication regarding presence of the molecule in the sample.

According to some embodiments, the biochip comprises at least two chambers, each chamber comprising a genetically modified bacteria expressing a receptor capable of binding a different molecule associated with the same or a different gastrointestinal disease or disorder.

According to some embodiments, the biochip comprises at least two chambers, configured to periodically receive a biological sample, e.g. every hour, every day, every week, every month every 6 months or every year. Each possibility is a separate embodiment.

According to some embodiments, the processing module is configured to provide a clinical indication based on an integrated analysis of the outputs obtained from each of the at least two chambers.

According to some embodiments, there is provided a method for evaluating the presence of a molecule associated with a gastrointestinal disease or disorder, the method comprising:
(a) providing a biochip comprising one or more chambers, each chamber comprising a synthetic biosensor module and a detection module, wherein the synthetic biosensor module comprises a genetically modified bacteria expressing a receptor capable of binding the molecule, and a reporter gene, wherein expression of the reporter gene is induced by the binding of the molecule to the receptor; and wherein the detection module comprises a reporter gene substrate, wherein interaction between the expressed reporter gene and the reporter gene substrate produces an output signal indicative of presence of the molecule;
(b) contacting the biochip with a biological sample from a subject in need thereof;
(c) detecting the output signal produced by the biochip in response to said contacting,
(d) providing an indication related to the gastrointestinal disease or disorder, based on the detected output signal.

According to some embodiments, the level of the molecule corresponds to the stage of the gastrointestinal disease or disorder.

According to some embodiments, the molecule is a quorum-sensing molecule. According to some embodiments, the quorum-sensing signaling molecule is an N-acyl-L-homoserine lactone. According to some embodiments, the quorum-sensing signaling molecule is an autoinducer 2.

According to some embodiments, the molecule is a siderophore. According to some embodiments, the molecule is an inflammation marker, such as but not limited to lipocalin and/or calprotectin.

According to some embodiments, the biochip comprises at least two chambers, each chamber comprising a genetically modified bacteria expressing a receptor capable of binding a different molecule associated with the same or a different gastrointestinal disease or disorder.

According to some embodiments, the indication related to the gastrointestinal disease or disorder is provided based on an integrated analysis of the outputs obtained from each of the at least two chambers.

According to some embodiments, the gastrointestinal disease or disorder is inflammatory bowel disease and/or C. difficile infection. According to some embodiments, the inflammatory bowel disease is Crohn's disease. According to some embodiments, the inflammatory bowel disease is ulcerative colitis.

According to some embodiments, the biological sample is selected from the group consisting of: urine, saliva, mucus and stool.

According to some embodiments, there is provided a method for evaluating the presence of a molecule associated with a pathogenic bacteria in a food and/or beverage sample intended for consumption, the method comprising:
(a) providing a biochip comprising one or more chambers, each chamber comprising a synthetic biosensor module and a detection module, wherein the synthetic biosensor module comprises a genetically modified bacteria expressing a receptor capable of binding the molecule, and a reporter gene, wherein expression of the reporter gene is induced by the binding of the molecule to the receptor; and wherein the detection module comprises a reporter gene substrate, wherein interaction between the expressed reporter gene and the reporter gene substrate produces an output signal indicative of presence of the molecule in the sample;
(b) contacting the biochip with the food and/or beverage sample;
(c) detecting the output signal produced by the biochip in response to the contacting,
(d) providing an indication related to the safety of the food and/or beverage from which the sample was obtained, based on the detected output signal.

According to some embodiments, the molecule is a quorum-sensing molecule. According to some embodiments, the quorum-sensing signaling molecule is an N-acyl-L-homoserine lactone. According to some embodiments, the quorum-sensing signaling molecule is an autoinducer 2. According to some embodiments, the quorum sensing molecule is selected from C4-HSL, C6-oxo-HSL, 3-oxo-C12-HSL, C12-HSL, C10-HSL, 3-oxo-C10HSL, AI-2, or combinations thereof.

According to some embodiments, the detector is a C4-HSL receptor, a C6-oxo-HSL, 3-oxo-C12-HSL, C12-HSL receptor, C10-HSL receptor, 3-oxo-C10-HSL receptor, a QscR, AI-2 receptor or any combination thereof.

According to some embodiments, the molecule is a siderophore. According to some embodiments, the molecule is an inflammation marker, such as but not limited to lipocalin and/or calprotectin.

According to some embodiments, the biochip comprises at least two chambers, each chamber comprising a genetically modified bacteria expressing a receptor capable of binding a different molecule associated with the same or different pathogenic bacteria. According to some embodiments, the biochip comprises at least two chambers, each chamber configured to sequentially receive food and/or beverage samples.

According to some embodiments, the indication related to the safety of the food/beverage from which the sample was obtained is provided based on an integrated analysis of the outputs obtained from each of the at least two chambers.

According to some embodiments, there is provided a sampling tube comprising the herein disclosed biosensor. According to some embodiments, the sampling tube may be associated with a main tube through used for flow through of a food or beverage product. According to some embodiments, the fluid flow communication between the main tube and the sampling tube may be governed by a one-way valve allowing flow of the food and or beverage product into the sampling tube, while preventing backflow from the sampling tube into the main tube. According to some embodiments, the opening of the one-way valve may be manually controlled. According to some embodiments, the one-way valve may be configured to automatically open, e.g. at predetermined timepoints, e.g. based on signal obtained from a control unit.

Other objects, features and advantages of the present invention will become clear from the following description, examples and drawings.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the Figures:

FIG. 1A is a scheme illustrating synthetic the herein disclosed biosynthetic sensor and quantitative detection modules, according to some embodiments.

FIG. 1B is a scheme illustrating optional operation of the herein disclosed biochip, according to some embodiments.

FIG. 1C is a scheme illustrating optional operation of the herein disclosed biochip, according to some embodiments.

FIG. 1D present results obtained using the herein disclosed biosynthetic sensor module, here a biosynthetic sensor module configured to detect C4-HSL producing bacteria. Plates were seeded with a genetically modified E. coli (pEC61.5), expressing the RhIR N-acyl-L-homoserine lactones (AHL) response regulator and the β-galactosidase enzyme under a AHL-C4-HSL sensitive promotor. In the absence of C4-HSL, no β-galactosidase expression was induced (upper panel). However, upon detection (exposure) to exogenous a C4-HSL producing bacteria, β-galactosidase expression was induced leading to formation of bluish phenotype, lower panel).

FIG. 2A is a scheme illustrating the general principle of the BioChip disclosed herein, containing electrochemical cells, each comprising a genetically modified bacteria, capable of acting as sensor of a specific quorum sensing signal molecule.

FIG. 2B is a scheme illustrating a BioChip comprising several electrochemical cells, each cell contains unique synthetic (genetically modified) bacteria, capable of detecting different molecules associated with gastro intestinal diseases and/or with pathogenic bacteria.

FIG. 2C is a block diagram of an optional electric circuit within the BioChip.

FIG. 3A is an exemplary graph of a chronoamperometric measurement demonstrating the correlation between the intensity of an electric current and the concentration of a molecule, here (C4-HSL), obtained using a synthetic biosensor module comprising E. coli genetically modified to express B-galactosidase, when exposed to C4-HSL.

FIG. 3B is an exemplary graph of a chronoamperometric measurement demonstrating the correlation between the intensity of an electric current and the concentration of a molecule (here C4-HSL), obtained using a synthetic biosensor module comprising E. coli genetically modified to express β-galactosidase in a C4-HSL dependent manner, after exposure to conditioned culture media of the C4-HSL producing P. aeruginosa.

FIG. 3C is an exemplary graph of a chronoamperometric measurement demonstrating the correlation between the intensity of an electric current and the concentration of a molecule (here AI2), obtained using a synthetic biosensor module comprising E. coli genetically modified to express β-galactosidase under an AI-2 dependent promotor, using a synthetic biosensor comprising E. coli genetically modified to express β-galactosidase after exposure to E. coli genetically modified to express luxl from Vibrio fishery.

FIG. 3D is an exemplary graph of a chronoamperometric measurement demonstrating the correlation between the intensity of an electric current and the concentration of a molecule (here AI2), obtained using a synthetic biosensor module comprising E. coli genetically modified to express β-galactosidase in a AI-2 dependent manner, using a synthetic biosensor comprising E. coli genetically modified to express B-galactosidase after exposure to Staphylococcus aureus.

FIG. 4 shows electrical detection of two different molecules, here AI-2 and C4-HSL molecules in a bacterial sample containing S. aureus and P. aeruginosa bacteria. Biosynthetic sensor modules configured to detect each of the molecules AI-2 and C4-HSL, where loaded with samples containing different ratios of S. aureus and P. aeruginosa. The electrochemical cell containing: bacterial sample (S. aureus and P. aeruginosa) (50%), 1.6 mg/ml PAPG substrate (10%) and the E. coli AI-2/C4-HSL biosensor (40%).

FIG. 5 shows chronoamperometric measurement obtained when exposing a C4-HSL dependent biosensor to various test conditions.

FIG. 6 shows chronoamperometric measurement obtained when exposing a C4-HSL dependent biosensor, a 3-oxo-C12-HSL dependent biosensor and a QscR based biosensor P. aeruginosa condition media.

FIG. 7A shows chronoamperometric measurement obtained when exposing a C4-HSL dependent biosensor to various P. aeruginosa biofilm concentrations.

FIG. 7B shows the bacterial count in the biofilm measured in FIG. 7A.

FIG. 7C shows chronoamperometric measurement obtained when exposing a C4-HSL dependent biosensor to a P. aeruginosa biofilm at various time points after adding PAPG.

FIG. 8 shows chronoamperometric measurement obtained when exposing a biosensor to various P. aeruginosa biofilm concentrations.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

As used herein and in the appended claims, the singular forms “a”, “an”, and “the” include plural referents unless the context clearly dictates otherwise. At times, the claims and/or disclosure may include terms such as “a plurality,” “one or more,” or “at least one;” however, the absence of such terms is not intended to mean, and should not be interpreted to mean, that a plurality is not conceived.

A “plurality” as used herein refers to more than one. For example, a plurality of compounds may be two, three, four, five, or more. Each possibility is a separate embodiment.

The term “about” when used before a numerical designation indicates approximations which may vary by (+) or (−) 10%, 5%, or 1%.

“Comprising” or “comprises” is intended to mean that the devices, compositions, and methods include the recited elements, and may additionally include any other elements.

According to some embodiments, the term “comprising” may be substituted with the term “Consisting essentially of” or consisting of”.

“Consisting essentially of” shall mean that the devices, compositions, and methods include the recited elements and exclude other elements of any essential significance to the combination for the stated purpose. Thus, a device, composition, or method consisting essentially of the elements as defined herein would not exclude other materials or steps that do not materially affect the basic and novel characteristic(s) of the claimed invention.

“Consisting of” shall mean that the devices, compositions, and methods include the recited elements and exclude anything more than trivial or inconsequential elements or steps. Embodiments defined by each of these transitional terms are within the scope of this disclosure.

According to some embodiments, there is provided a biochip including one or more chambers/compartments/cell, each chamber including a synthetic biosensor module and a detection module. The synthetic biosensor module includes genetically modified bacteria configured to detect a specific marker molecule, (optionally, each chamber including a bacteria configured to detect a different marker molecule) and, in response thereto, trigger expression of a reporter gene. The detection module includes one or more sensors/detectors configured to detect the presence of the reporter gene, preferably in a quantitative manner.

According to some embodiments, each chamber may be separated from the external environment and/or from a neighboring chamber by a semipermeable membrane, which confines the biochip therewithin, yet allows entry of the molecules. According to some embodiments, each chamber may be concealed from its neighboring chamber to prevent flow therebetween.

According to some embodiments, the marker molecules are found in a biological sample, such as but not limited to a stool sample, a urine sample, a saliva sample or combinations thereof. Each possibility is a separate embodiment. Advantageously, the biochip may enable detection of the marker molecule in a sample while requiring a short incubation time only (typically 1-5 hours, 1-4 hours, 2-4 hours or 2-3 hours. Each possibility is a separate embodiment) as opposed to the 24-48 culturing typically required. In addition, the herein disclosed sensor may enable detecting low levels of molecules (e.g. below 10\hat{ }6 or below 10\hat{ }5 bacteria)], thereby allowing early detection of diseases.

According to some embodiments, the marker molecules are found in food stuff, water supplies, cosmetic products and the like. Advantageously, the biochip may enable detection of biofilm formation in a flow/through sample, thereby a) obviating the need from manually collecting a sample and casing periodic sampling; b) requiring short incubation time only (typically 1-5 hours, 1-4 hours, 2-4 hours or 2-3 hours. Each possibility is a separate embodiment), thereby obviating the need for culturing of the sample, which typically is done for 24-48 hours; and c) enabling detection of low levels of bacteria, preferably below upper health-associated limits.

According to some embodiments, the bacteria include, optionally as a result of genetic engineering, a receptor configured to bind the molecule. According to some embodiments, as a result of the binding, a reporter gene, such as, but not limited to β-galactosidase, is expressed by the bacteria. According to some embodiments, the quantitative detection module of the biochip includes a substrate, such as p-aminophenyl-β-D-galactopyranoside (PAPG), which when hydrolyzed forms PAP, which PAP in the presence of an external input voltage, generates an output current signal. According to some embodiments, each of the chambers has its own embedded readout electronics, such that the electric response can be associated with the molecule activating it.

According to some embodiments, the bacteria may be further genetically modified to delete the endogenous version of the reporter gene. As a non-limiting example, the endogenous B-galactosidase of E. coli may be deleted in order to prevent noise. According to some embodiments, the receptor of the molecule may be endogenous. According to some embodiments, the bacteria may be genetically modified to express the receptor of a specific marker molecule. According to some embodiments, a bacterial strain may be generated, in which strain the gene that synthesizes the marker molecule has been deleted. Advantageously, such strain may be and used to detect the signal as it has an intact receptor system.

According to some embodiments, the detection module detects electrical signals on a nanoampere scale, thereby allowing detection of very low levels (nM concentrations) of the molecule of interest as wells as subtle changes in the levels thereof.

According to some embodiments, the biochip may, optionally wirelessly, transmit a signal, such as a radiofrequency signal to an external receiver. According to some embodiments, the external receiver or other integrated system may supply power to the biochip, read the signal, and then process and transmit the data. According to some embodiments, the external receiver may be a mobile phone with a dedicated App. According to some embodiments, the external receiver may be a dedicated electrochemical reader.

Advantageously, the herein disclosed biochip may include an array/plurality of chambers, e.g. 2, 3, 4, or more chambers. Each possibility is a separate embodiment. According to some embodiments, the plurality of chambers include a respective plurality of genetically modified bacteria, each configured to detect a different marker molecule.

According to some embodiments, identification of more than a single marker molecule may enable a more precis identification of the source of an illness. As a non-limiting example, while one molecule may in some instances only allow identification of abnormal gram-negative bacteria or an abnormal level of a gram-negative bacteria, identification of additional molecules may further provide an indication as to the strain of the gram-negative bacteria.

Moreover, identification of more than one marker molecule may enable detecting changes in the ratio between different marker molecules. As a non-limiting example, identification of more than one molecule may enable detecting changes in the ratio between different molecules which change may be indicative of a change in the bacterial composition of the microbiome. Advantageously, this may allow early diagnosis of gastrointestinal diseases/abnormalities as well as their severity and/or changes therein.

According to some embodiments, the system may include a processing module configured to analyze the output obtained from a plurality of chambers and to apply an AI algorithm thereon. According to some embodiments, the AI algorithm may be a machine learning algorithm. According to some embodiments, the machine learning algorithm may be supervised machine learning algorithm. According to some embodiments, the training set of the machine learning algorithm may be performed on molecules identified in samples obtained from subjects with a known gastrointestinal disorder as well as from generally healthy subjects.

According to some embodiments, the marker molecule is a quorum sensing molecule. According to some embodiments, the quorum sensing molecule is an autoinducer (AI) typically associated with gram-positive quorum sensing or a N-acyl homoserine lactone, typically associated with Gram-negative quorum sensing. According to some embodiments, the quorum sensing molecule is selected from C4HSL, C6-oxo-HSL, 3-oxo-C12-HSL, C12-HSL, 10-HSL, 3-oxo-C10HSL, AI-2, or combinations thereof.

According to some embodiments, the marker molecule may be a disease activity marker, such as, but not limited to lipocalin-2 and calprotectin (upregulated in inflammatory bowel disease.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

### EXAMPLES

### Example 1: The Synthetic Biosensor Module

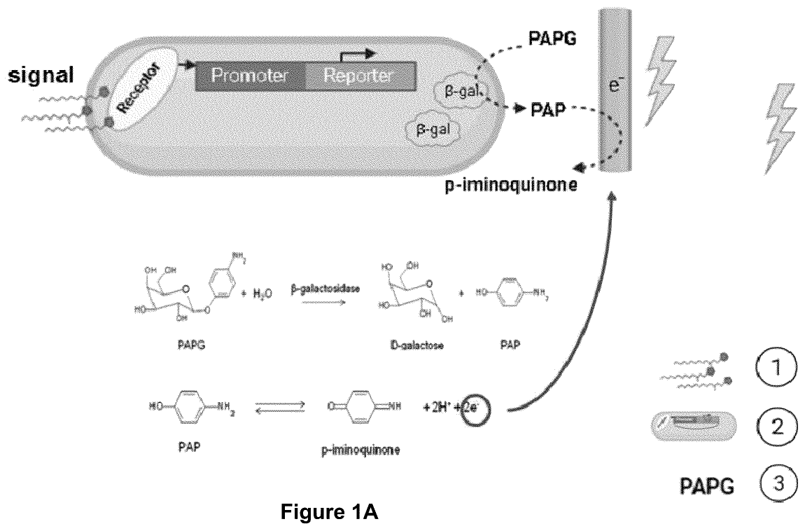

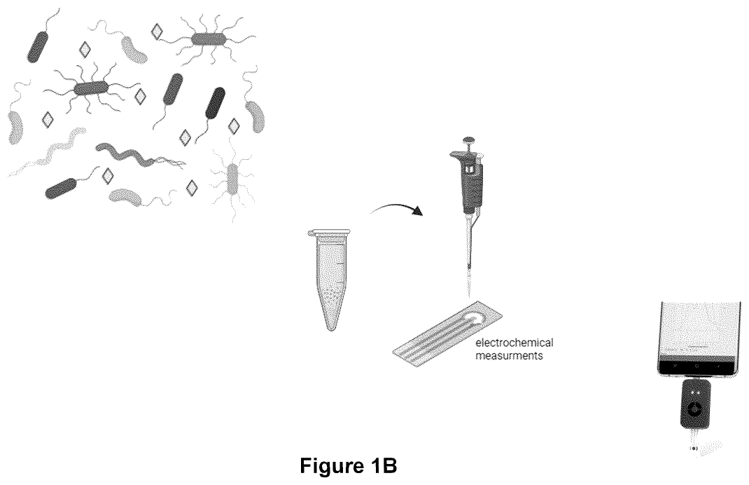

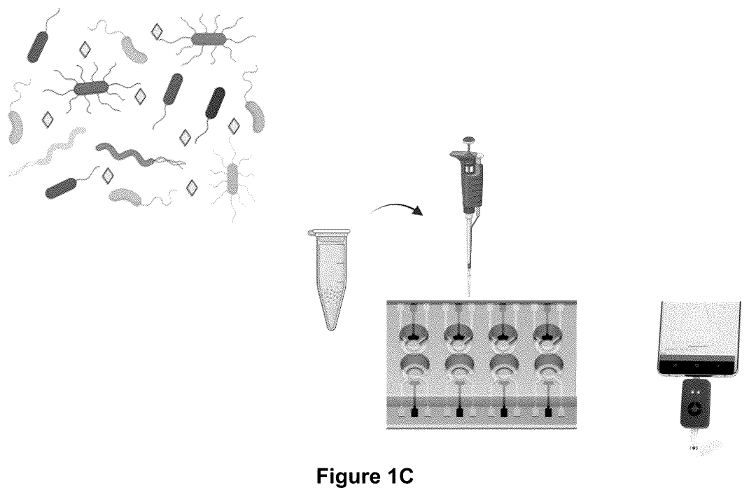

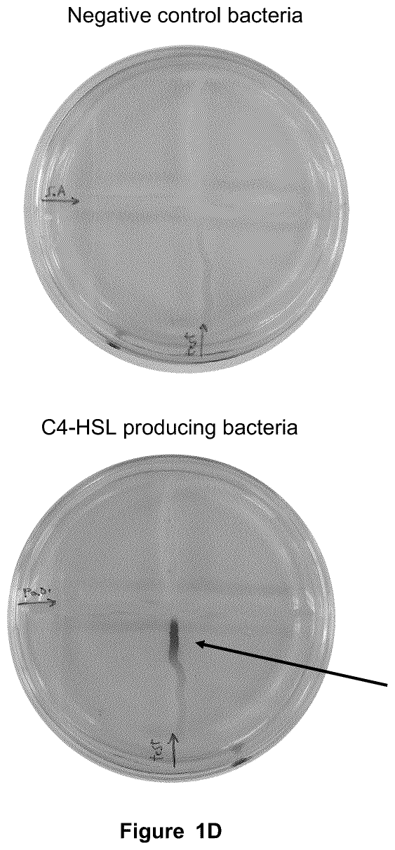

Synthetic biology was employed to engineer a biomarker related cascade, as illustrated in FIG. 1A, by cloning key players: β-galactosidase under inducible promotors. To this end, several plasmids were constructed and transformed into E. coli, wherein each plasmid contained different genes encoding specific receptors. The constructs were developed from available plasmids. FIG. 1A illustrates the three key components required by the synthetic biosensor: 1) marker molecules such as, but not limited to lipocalin or Calprotectin, secreted by the bacteria to be detected; 2) specific receptor encoding gene (‘receptor gene’), and 3) a reporter gene that upon formation of a receptor-marker molecule complex is triggered (by direct or indirect binding) of the receptor-marker molecule complex to the promotor of the reporter gene (here illustrated as B-galactosidase. B-galactosidase reacts with the substrate PAPG, releasing the redox mediator PAP, which is then oxidized at an electrode, and current signal is generated.

Based on the engineered bacterial system described above, whole-cell based biochip sensor technology containing biochemical and electrical systems for sensing specific signals, and configured to diagnose microbiome related disorders was developed. An exemplary design of the whole-cell based biochip sensor are illustrated in FIG. 1B and FIG. 1C. In essence, the biochip includes a miniaturized electrochemical cell, (FIG. 1B) or an array of miniaturized electrochemical cells (FIG. 1C), bottom panel, where each cell may be separated from the external environment by a semipermeable membrane, which confines the bacteria biosensors therewithin, yet allows entry of molecules into the cell. Each cell includes a suspension comprising genetically engineered bacteria, specifically tailored to produce β-gal following uptake of specific marker molecules (as described above and illustrated in FIG. 1A). For electrochemical detection of the marker molecules, each electrochemical cell may include substrate configured to produce an electric signal, upon interacting with a reporting molecule. As exemplified in FIG. 1A, p-aminophenyl-β-D-galactopyranoside (PAPG), a synthetic analogue of β-gal's natural substrate lactose, can be used. This substrate once hydrolyzed (catalyzed by β-gal) generates an electroactive PAP product (the electro-oxidation of PAP at an electrode surface). Each cell has its own embedded readout electronics, thereby enabling accurate association of a specific electric response with a specific marker molecule.

According to some embodiments, in use, a sample, such as a stool sample, containing variety of bacteria at various concentration, may be obtained. The sample may be processed (e.g. suspended in an aqueous solution) and loaded on the biochip, as shown in FIG. 1B. The electrical signals may then be transmitted/provided to an external receiver/reader, in FIG. 1B illustrated as a mobile device.

The ability of the herein disclosed biochip to detect marker molecules was demonstrated with a genetically modified strain of E. coli (pEC61.5), expressing an N-acyl-L-homoserine lactones (AHL) receptor. Upon exposure to a C4-HSL producing bacteria, β-galactosidase expression was induced, leading to 5-bromo-4-chloro-3-indolyl-B-D-galactopyranoside (X-Gal) hydrolysis, resulting in blue coloring of bacteria culture/colonies (FIG. 1D).

### Example 2: The Quantitative Detection Module

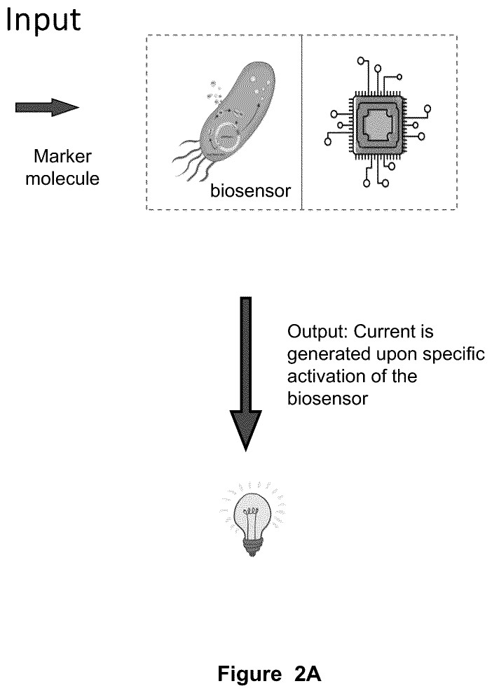

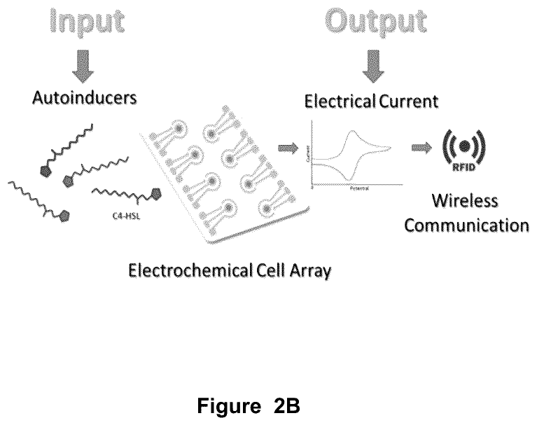

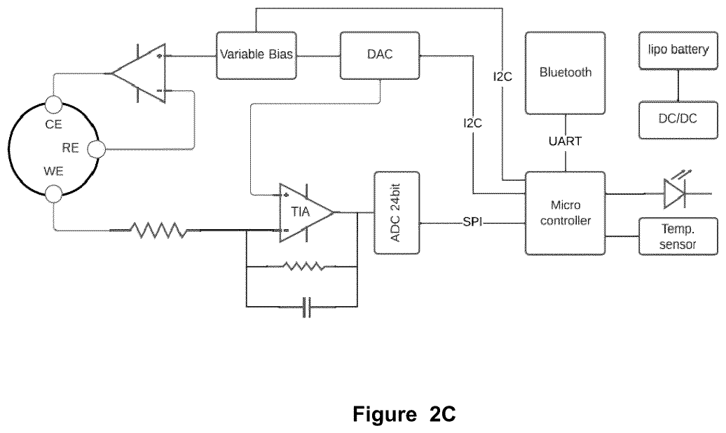

A scheme illustrating the general principle of the BioChip disclosed herein, containing electrochemical cells, each comprising genetically modified bacterium, namely, a synthetic biosensor module, and a microelectronic detection module configured to sense electrical signals, process the signal, if required, and transmit them to an external unit, namely, a receiver, is shown in FIG. 2A. The biosensor acts as a sensing element and triggers a cascade mechanism in response to exposure to the marker molecule, wherein the cascade mechanism leads to generation of electrical current in an electrochemical cell. According to some embodiments, each BioChip comprises a plurality of electrochemical cells, as illustrated in FIG. 2B, which can be adapted for simultaneous detection of a variety of bacterial strains in the crowded microbiome environment.

A schematic illustration of an optional block diagram of an electric circuit, which may be used in the context of the BioChip disclosed herein, is shown in FIG. 2C. Each electrochemical cell, or sensor may be connected to analog front end via 3 electrodes. A processor adapted to control the sensor voltage levels (@ CE and RE electrodes) via an amplifier, digital to analog converter and a variable bias may be further included. The processor is configured to set the required DAC output voltage level and to control on the fly a variable percentage of this voltage to be supplied to an amplifier. The voltage applied on the strip, in a case of chemical reaction (interaction with a marker molecule), results in a current flowing throw working electrode (WE) to a load resistor and a trans impedance amplifier. A transimpedance amplifier (TIA) circuit is then adapted convert the current signal into a voltage and amplify it by a factor which can be set via the microcontroller. The amplified signal is configured to enter a 24-bit analog-to-digital converter (ADC) which converts the analog signal into a digital level that can be read by the microcontroller via SPI protocol. The electro chemical measured current can be transmitted, e.g. via Bluetooth low energy module to an external device (tablet/PC GUI). Via such wireless interface, a user can configure and control the electrochemical sensor parameters, operation, and view the sensor voltage/current levels in Real Time. A temperature sensor can be used to calibrate the sensor's readings in various conditions. The sensor may be battery operated and may have an additional DC2DC converter and optional charging circuitry.

### Example 3: Biochip Measurements

In order to evaluate the ability of different synthetic biosensors to detect different marker molecules in a quantitative manner, chronoamperometric measurements were performed using an electrochemical cell containing E. coli genetically modified to express β-galactosidase in a C4-HSL/AI-2)-dependent manner (E. coli (c4-HSL) detector); PAPG substrate (1.6 mg/ml) and various sources of C4-HSL co-incubated for 3 h.

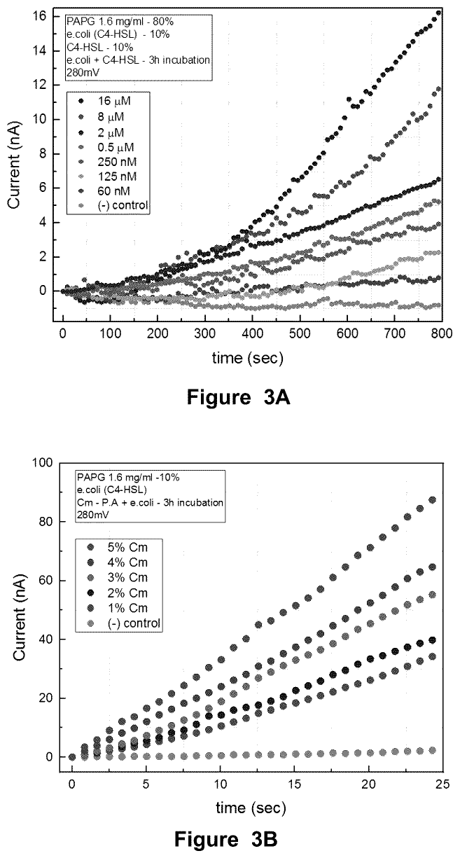

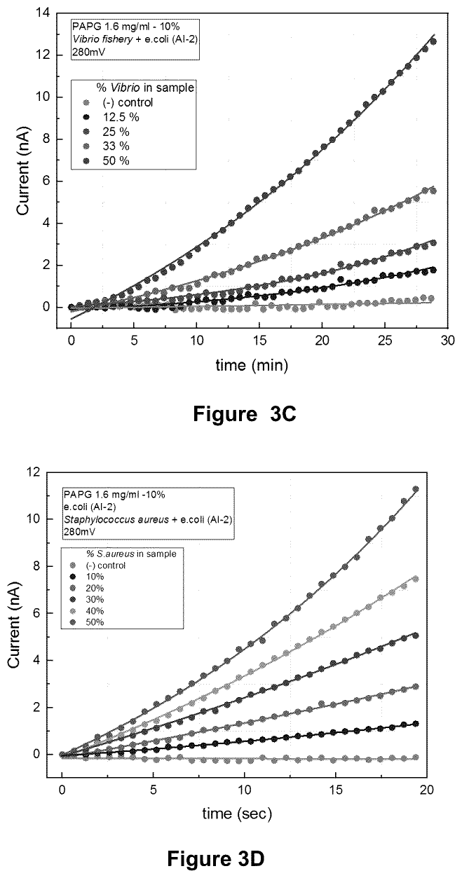

As seen, a clear and stable electric current signal was produced immediately after exposure of the synthetic biosensor to the tested molecule indicating the production of β-galactosidase (B-gal) by the biosensor bacteria upon exposure to the marker molecule: FIG. 3A—10% purified C4-HSL at indicated concentrations, 80% PAPG and 10% detector, FIG. 3B—different concentrations of condition medium of P. aeruginosa which naturally secret C4-HSL, FIG. 3C—different concentrations of V. fischiri (A12 producing E. coli) and FIG. 3D different concentrations of the A12 producing S. aureus, thereby demonstrating the feasibility and sensitivity of the biochip.

### Example 4: Biochip Measurements of Complex Samples

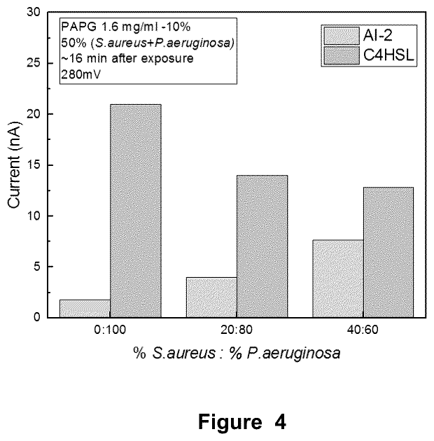

In order to evaluate the ability of the herein disclosed biochip to reliably identify bacteria in mixed samples and their relative ratios, biosynthetic sensor modules configured to detect each of the molecules AI-2 and C4HSL, where loaded with samples containing different ratios of S. aureus and P. aeruginosa, which secrete the AI-2 and C4HSL molecules respectively. As seen from FIG. 4, a clear correlation between the relative value of the current signal and the ratio between the bacteria. This advantageously demonstrate that the ability of the herein disclosed biochip to identify different molecules in mixed samples as well as to reliably detect their relative abundance.

### Example 5: Biochip Measurements Using P. aeruginosa-C4-HSL Sensor

The efficacy of the herein disclosed biosensor was further evaluated using a different detector namely P. aeruginosa genetically modified to express β-galactosidase in a C4-HSL dependent manner (JP2 rh1A).

The following conditions were tested:
1. “PBS no detector” (negative control)
2. A P. aeruginosa genetically modified to be unable to produce C4-HSL and 3-oxo-C12-HSL expression “MW1” in absence of a detector-“MW1 no detector” (second negative control).
3. Condition medium of MW1-“CM MW1” (third negative control).
4. MW1 with detector-“MW1” (fourth negative control).
5. Condition media of a bacteria which does not produce 3-oxo-C12-HSL “CM dlasl”. 3-oxo-C12-HSL induces C4-HSL and its absence thus reduces C4-HSL levels (low C4-HSL control).
6. Bacteria which does not produce 3-oxo-C12-HSL “dlasl”.
7. Condition media of wild type P. aeruginosa “CM PAO1” in the presence of the detector.
8. Wild type P. aeruginosa “PAO1” in the presence of the detector.

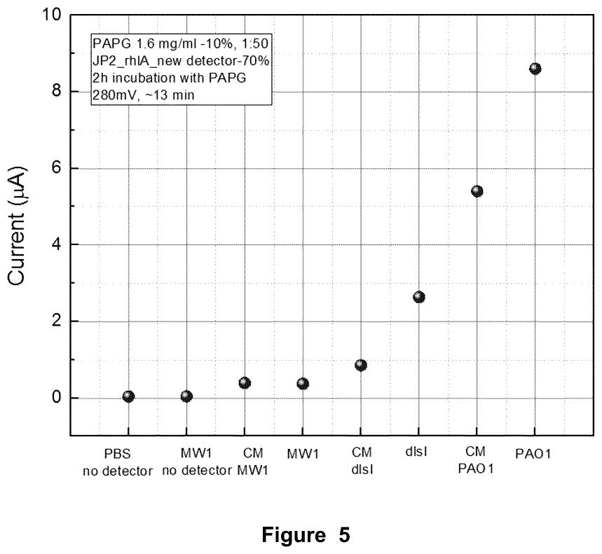

As can be seen from FIG. 5, essentially no signal was obtained for the negative controls 1-4, and, as expected, only a week signal was obtained for the 3-oxo-C12-HSL mutant. However, presence of P. aeruginosa, whether the bacteria or its condition media only, resulted in a strong and significant induction of current flow, thus confirming the efficacy of the herein disclosed biosensor.

### Example 6: Biochip Measurements Using Various P. aeruginosa Biosensors

In order to further evaluate the herein disclosed biosensor, various detectors based on P. aeruginosa were generated, namely:
1. P. aeruginosa genetically modified to express B-galactosidase in a C4-HSL dependent manner (as in Example 5);
2. P. aeruginosa genetically modified to express β-galactosidase in a C12-oxo-HSL dependent manner; and
3. P. aeruginosa genetically modified to express β-galactosidase in a QscR dependent manner (i.e., when either of the quorum sensing molecules 3-oxo-C12-HSL, C12-HSL, 3-oxo-C10-HSL, C10-HSL bind the QscR receptor.

The detectors were incubated with P. aeruginosa condition media (or left untreated (control) and readings were made over time after addition of the PAPG substrate.

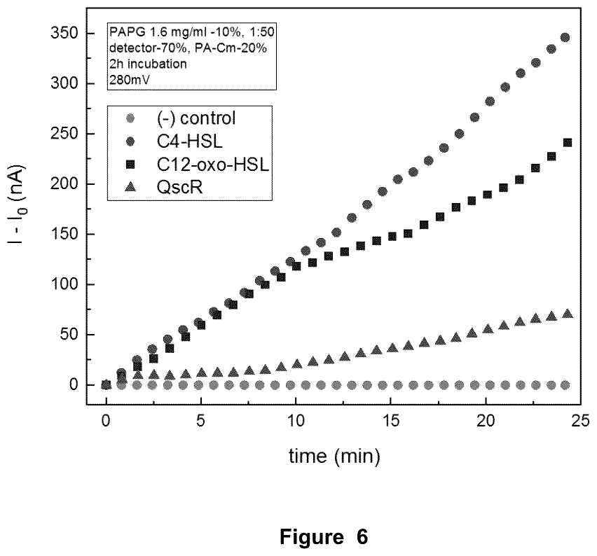

As can be seen from FIG. 6, significant increases in current flow were obtained for all three detectors after exposure to the condition media after about 15 min incubation with PAPG.

### Example 7: Biofilm Detection Using a P. aeruginosa C4-HSL Biosensor

In order to investigate the ability to detect biofilm formation during flow, biofilm was formed by growing P. aeruginosa in varying concentrations of growth media (0.1, 1 and 10%).

Flow-through from the biofilm was collected and mixed with the rhlA biosensor (P. aeruginosa genetically modified to express β-galactosidase in a C4-HSL dependent manner) and the mixture incubated for 3 h.

90 μl of the mixture was then mixed with 10 μl PAPG (detector) and electrochemical measurement performed at different timepoint.

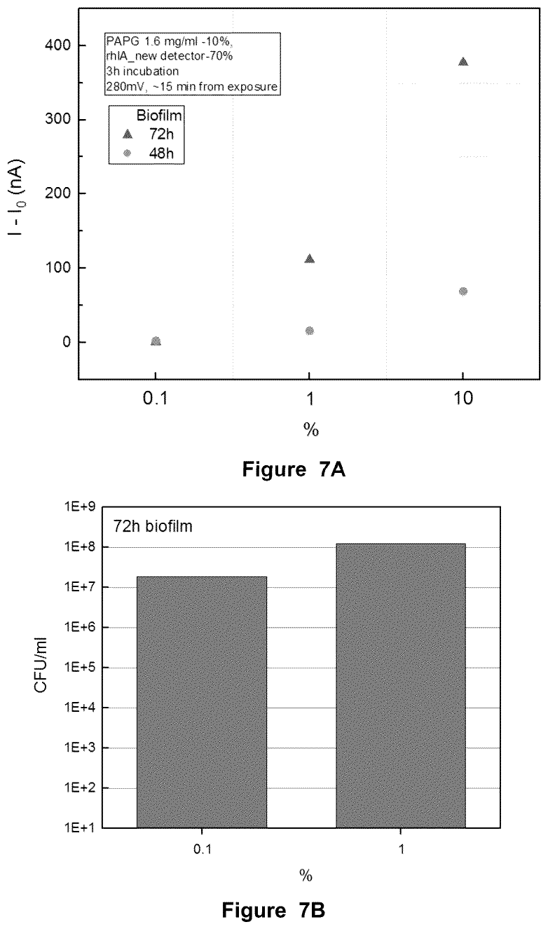

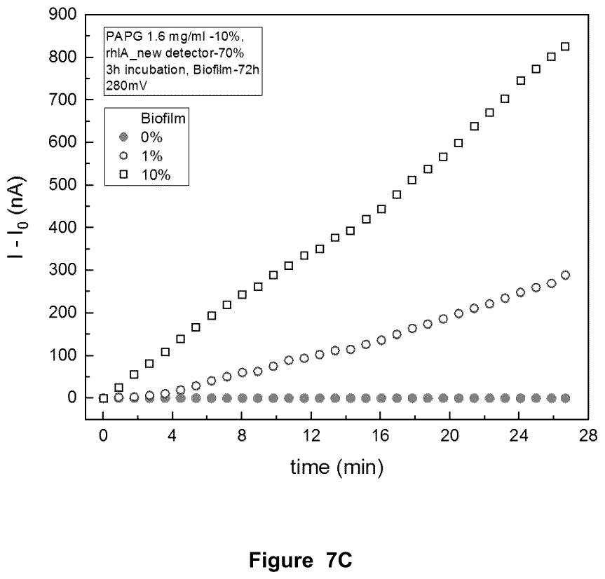

FIG. 7A, shows the change in current flow obtained as a result of mixing P. aeruginosa biofilm obtained by growing the P. aeruginosa in 0.1%, 1% and 10% growth media for 48 or 72 h with the rhIA biosensor, each reading normalized to the reading obtained in the absence of biofilm. As seen from FIG. 7A, exposing the biosensor to biofilm obtained after growing P. aeruginosa in 1% growth media for 72 h, corresponding to a bacterial count of about 1.00E+08 cfu/ml (FIG. 7B), caused a significant increase in current flow, 15 minutes after exposure to PAPG, thus demonstrating the ability of the herein disclosed biosensor to measure biofilm formation in a flow through setting in a rapid and reliable manner.

Furthermore, as seen from FIG. 7C, the sensitivity of the biosensor can advantageously be adjusted based on the time of incubation with PAPG, indicating that longer PAPG incubation (such as about 20-25 minutes can enable detection of lower bacterial counts.

### Example 8: Biofilm Detection Using a P. aeruginosa QscR Biosensor

In order to further investigate the ability to detect biofilm formation during flow, biofilm was formed by growing P. aeruginosa in varying concentrations of growth media (0.1, 1 and 10%).

Flow-through from the biofilm was collected and mixed with the QscR biosensor (P. aeruginosa genetically modified to express B-galactosidase when either of the quorum sensing molecules, 3-oxo-C12-HSL C12-HSL, 3-oxo-C10-HSL, C10-HSL bind the QscR receptor) and the mixture incubated for 3 h.

90 μl of the mixture was then mixed with 10 μl PAPG (substrate) and electrochemical measurement performed at different timepoint.

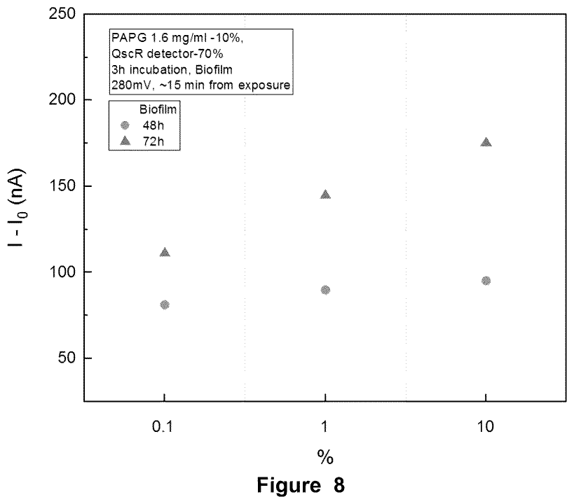

FIG. 8, shows the change in current flow obtained as a result of mixing P. aeruginosa biofilm obtained by growing the P. aeruginosa in 0.1%, 1% and 10% growth media for 48 or 72 h with the QscR biosensor, each reading normalized to the reading obtained in the absence of biofilm. As seen from FIG. 8, exposing the biosensor to biofilm obtained after growing P. aeruginosa in 0.1% growth media for 72 h, corresponding to a bacterial count of about 1.00E+07 cfu/ml, caused a significant increase in current flow 15 minutes after exposure to PAPG, thus demonstrating the ability of the herein disclosed biosensor to measure biofilm formation in a flow through setting in a rapid and reliable manner and at relatively low bacterial concentrations.

### Example 9: Biochip Measurements with Disease Activity Marker Molecules

In order to evaluate the ability of the herein disclosed biosensor to detect other marker molecules in a quantitative manner, chronoamperometric measurements are performed using an electrochemical cell containing bacteria genetically modified to express β-galactosidase in a calprotectin and/or Lipocalin-2 dependent manner (or other marker molecule associated with a GI disease)); PAPG substrate (1.6 mg/ml) and various sources of calprotectin and/or Lipocalin-2 co-incubated for 3 h.

### Example 10: Biochip for Detection of IBD in Stool Samples

In order to evaluate the ability of the herein disclosed biosensor to detect calprotectin and/or Lipocalin-2 or other IBD associated marker molecule in stool samples, chronoamperometric measurements are performed using an electrochemical cell containing bacteria genetically modified to express β-galactosidase in a calprotectin and/or Lipocalin-2 dependent manner detector); PAPG substrate (1.6 mg/ml) and stool samples obtained from IBD patients, patients suffering from irritable bowel syndrome and control subjects.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

Unless specifically stated otherwise, as apparent from the disclosure, it is appreciated that, according to some embodiments, terms such as “processing”, “computing”, “calculating”, “determining”, “estimating”, “assessing”, “gauging” or the like, may refer to the action and/or processes of a computer or computing system, or similar electronic computing device, that manipulate and/or transform data, represented as physical (e.g. electronic) quantities within the computing system's registers and/or memories, into other data similarly represented as physical quantities within the computing system's memories, registers or other such information storage, transmission or display devices.

Embodiments of the present disclosure may include apparatuses for performing the operations herein. The apparatuses may be specially constructed for the desired purposes or may include a general-purpose computer(s) selectively activated or reconfigured by a computer program stored in the computer.

1. A biochip for detection of a marker molecule in a sample, the biochip comprising one or more chambers, each chamber comprising a synthetic biosensor module and a detection module,
wherein the synthetic biosensor module comprises a genetically modified bacteria expressing a receptor capable of binding the marker molecule, and a reporter gene,
wherein expression of the reporter gene is induced by the binding of the marker molecule to the receptor; and
wherein the detection module comprises a reporter gene substrate, wherein interaction between the expressed reporter gene and the reporter gene substrate produces an output signal indicative of presence of the marker molecule in the sample, and wherein marker molecule is associated with a gastrointestinal disease or disorder.

2. The biochip of claim 1, wherein the detection module is a quantitative detection module and/or qualitative detection module.

3. The biochip of claim 1, wherein an intensity and/or strength of the output signal is indicative of the concentration of the marker molecule in the sample.

4. The biochip of claim 1, comprising at least two chambers, each chamber comprising a genetically modified bacteria expressing a receptor capable of binding a different marker molecule.

5. The biochip of claim 4, wherein a ratio between the output signal of each of the at least two chambers is indicative of a ratio between different bacterial populations in the sample and/or of a severity and/or type of a gastrointestinal disease.

6. The biochip of claim 4, wherein combined outputs of the at least two chambers is indicative of a specific bacterial population in the sample and thus of a disease status of the patient.

7. The biochip of claim 1 comprising at least two fluidly separate chambers, each chamber configured to separately receive samples at different time points.

8. The biochip according to claim 1, wherein the output signal is an electric signal.

9. The biochip according to claim 1, wherein the reporting agent is selected from the group consisting of: beta-galactosidase, glutathione-S-transferase (GST), c-myc, 6-histidine (6×His), maltose binding protein (MBP), influenza A virus haemagglutinin (HA), and GALA.

10. The biochip according to claim 9, wherein the reporter gene is beta-galactosidase, and the reporter gene substrate is p-aminophenyl-β-D-galactopyranoside.

11. The biochip according to claim 1, further comprising a DNA probe encoding a transporter of said marker molecule.

12. A microbiome analysis system comprising
a biochip for detection of a marker molecule in a sample, the biochip comprising one or more chambers, each chamber comprising a synthetic biosensor module and a detection module, wherein the synthetic biosensor module comprises a genetically modified bacteria expressing a receptor capable of binding a marker molecule, and a reporter gene, wherein expression of the reporter gene is induced by the binding of the marker molecule to the receptor; and
wherein the detection module comprises a reporter gene substrate, wherein interaction between the expressed reporter gene and the reporter gene substrate produces an electric output signal;
a processing module configured to receive the output signal and to provide an indication regarding presence of the marker molecule in the sample.

13. The microbiome analysis system of claim 12, wherein the biochip comprises at least two chambers, each chamber comprising a genetically modified bacteria expressing a receptor capable of binding a different marker molecule.

14. The microbiome analysis system of claim 12, wherein the processing module is configured to provide a clinical indication/safety indication, based on an integrated analysis of the outputs obtained from each of the at least two chambers.

15. A method for evaluating the presence of a marker molecule, the method comprising:
a. providing a biochip comprising one or more chambers, each chamber comprising a synthetic biosensor module and a detection module, wherein the synthetic biosensor module comprises a genetically modified bacteria expressing a receptor capable of binding the marker molecule, and a reporter gene, wherein expression of the reporter gene is induced by the binding of the marker molecule to the receptor; and wherein the detection module comprises a reporter gene substrate, wherein interaction between the expressed reporter gene and the reporter gene substrate produces an output signal indicative of presence of the marker molecule;
b. contacting the biochip with a biological sample;
c. detecting the output signal produced by the biochip in response to said contacting,
d. providing an indication related to presence of the marker molecule in the sample, wherein the marker molecule is associated with a gastrointestinal disease or disorder.

16. The method of claim 15, wherein the indication is related to a gastrointestinal disease or disorder, and wherein the level of the marker molecule corresponds to the stage of the gastrointestinal disease or disorder.

17. The method according to claim 16, wherein the gastrointestinal disease or disorder is inflammatory bowel disease and/or C. difficile infection and wherein the inflammatory bowel disease is Crohn's disease or ulcerative colitis.

18. The method according to claim 15, wherein the biological sample is selected from the group consisting of: urine, saliva, mucus and stool.

19. The method of claim 15, wherein the biological sample is obtained from a food, a beverage, a water source or any combination thereof.
